(19) United States
(12) Patent Application Publication
(10) Pub. No.: US 2021/0121380 A1
(43) Pub. Date: Apr. 29, 2021

(54) Amino Acid Containing Oral Care Composition for Treating Caries By Reducing Lactic Acid Release in Oral Biofilms

(71) Applicant: 3M INNOVATIVE PROPERTIES COMPANY, St. Paul, MN (US)

(72) Inventors: Brenda Schmid, Markt Schwaben (DE); Melanie N. Hauke, Gilching (DE); Ingo R. Haeberlein, Weilheim (DE)

(21) Appl. No.: 17/250,306

(22) PCT Filed: Jul. 11, 2019
(86) PCT No.: PCT/IB2019/055937

§ 371 Date: Dec. 31, 2020

(30) Foreign Application Priority Data
Jul. 16, 2018 (EP) 18183601.6

(51) Int. Cl. A61K 8/44; A61Q 11/00

(52) U.S. Cl. CPC A61K 8/44; A61Q 11/00

(57) ABSTRACT

The invention relates to a composition for use in a method of treating caries by reducing the lactic acid release of lactic acid producing bacteria in an oral biofilm of a human being or animal, the composition comprising one or more amino acid(s) dissolved or dispersed in water, the amino acid(s) being selected from glycine, leucine, isoleucine, lysine, methionine, phenylalanine, serine, threonine, valine, tryptophan and mixtures thereof. The invention also relates to a kit of parts for use in a method of treating caries by reducing the caries activity or reducing the lactic acid release of lactic acid producing bacteria in an oral biofilm, the kit of parts comprising Part A comprising certain amino acid(s), Part B comprising water, and optionally Part C comprising an application device.

### FIELD OF THE INVENTION

The invention relates to a composition for use in a method of treating caries by reducing the lactic acid release of lactic acid producing bacteria in an oral biofilm of a living human or animal being. The composition comprises certain amino acid(s) dissolved or dispersed in water.

### BACKGROUND

Dental plaque, which may include bacteria such as Streptococcus mutatis, comprises a biofilm that forms on surfaces in the oral cavity. Dental plaque is at least partly responsible for dental caries, gingivitis, and periodontal diseases.

Bacteria in dental plaque metabolize carbohydrates (for example, simple sugars) in the mouth and produce acids that can etch tooth enamel, dentin, and cement. Dental plaque can serve as a substrate for the deposition of tartar or calculus. Build-up of dental plaque and calculus can lead to gingivitis and, ultimately, to periodontal disease.

A currently available method to remove dental plaque from teeth is mechanical removal with, for example, dental floss or a toothbrush. A toothbrush can aid in removing dental plaque from exposed surfaces of a tooth, and dental floss can aid in removing dental plaque from, for example, interproximal and subgingival surfaces. Proper and regular use of dental floss and a toothbrush can mechanically remove or reduce dental plaque, and can reduce the incidence of dental caries, gingivitis, and periodontal disease. Certain antimicrobial formulations are available (in the form of mouthwashes, rinses, and toothpastes, for example) to aid in the control and treatment of dental plaque, dental caries, gingivitis, and periodontal disease.

For many years prevention of caries treatment focused on prevention of dental plaque formation and dental plaque removal from tooth surfaces. Thus, various attempts have been made to provide an oral composition which is suitable for cleaning teeth and/or reducing the amount of plague and oral biofilm.

Daily clinical experience, however, indicates that most of the dental plaque prevalent in patient's mouth do not necessarily cause tooth demineralization, irrespective of the dietary behaviour of patients. Thus, resent clinical publications indicate, that the amount of plaque, which is permanently present in patient's mouth, is almost little correlated with patient's caries risk.

Meanwhile, it is also reported that dental caries results from an imbalance of the metabolic activity in the individual dental biofilm. This reflects the daily clinical experiences that only a small fraction of dental plaque might end up in tooth demineralization. Even more, certain studies with dental biofilm revealed that the bacterial composition of dental plaque does not necessarily indicate the prevailing metabolic (caries) activity of the individual dental plaque.

Attempts to influence the formation of biofilm and/or prevent the formation of plaque are described in various documents.

WO 2009/014907 A1 (Yang et al.) provides a dental composition comprising a compound of a certain formula or acceptable salt thereof for inhibiting the formation of biofilm. As an example of such a compound N-methyl-d-glucamine is mentioned.

WO 00/69890 A1 (Stromberg et al.) relates to an oligopeptide protecting against dental caries comprising two arginine residues selected from the group consisting of pentapeptide, hexapeptide, heptapeptide, octapeptide, nonapeptide and decapeptide.

CA 986 022 (Goldman et al.) describes a composition suitable for treating teeth for the prevention of plaque which comprises an aqueous solution of N-monochloroglycine buffered to a pH in the range of 9 to 11.5.

EP 0 224 599 A1 (National Patent Development Corp.) relates to a chemical solution useful in the removal of carious lesions or plaque, said solution being prepared immediately prior to use and formed by mixing DL-2-aminobutyric acid, glycine, sodium hydroxide, sodium chloride, sodium hypochloride in de-ionized water.

EP 0 711 543 A1 (Unilever) relates to an oral preparation having an anti-caries activity, comprising pyruvic acid or an orally-acceptable salt thereof and urea and/or arginine or a derivative thereof.

U.S. Pat. No. 9,750,670 B2 (Pan et al.) provides a composition for oral personal care comprising a tetrabasic zink-amino acid or trialkyl glycine-halide complex, cysteine in free or in orally or cosmetically acceptable salt form.

US 2007/0116831 A1 (Prakash et al.) describes a dental composition comprising an active dental substance such hydrogen peroxide, one high-potency sweetener such as rebaudioside A, and one sweet taste improving composition, such as an amino acid.

U.S. Pat. No. 4,339,432 (Ritchey et al.) relates to an oral mouthwash containing zinc and glycine. It is stated that the astringency of an oral mouthwash composition containing biologically active zinc ions may be reduced by the addition of glycine.

WO 2009/100263 A2 (Colgate) describes an oral care composition comprising the basic amino acid arginine if free or salt form together with an effective amount of a soluble calcium salt selected from calcium glycerophosphate and salts of soluble carboxylic acids. This composition is said to be effective in inhibiting or reducing the accumulation of plaque, reducing levels of acid producing (cariogenic) bacteria, remineralizing teeth and inhibiting or reducing gingivitis.

US 2009/0202456 A1 (Prencipe et al.) relates to a salt of arginine and one or more of the following conjugate acids: an acidic polymer, a conjugate acid of an anionic surfactant salt, a polyphosphoric or polyphosphonic acid or an acidic antimicrobial agent. The composition is said to be useful for protecting teeth by facilitating repair and remineralization, in particular to reduce or inhibit formation of dental caries, reduce or inhibit demineralization and promote remineralization of the teeth, reduce hypersensitivity of the teeth, and reduce, repair or inhibit early enamel lesions, e.g., as detected by quantitative light-induced fluorescence or electronic caries monitor.

### DESCRIPTION OF THE INVENTION

None of the attempts suggested so far is completely satisfying. As noted above, proper and regular use of dental floss and a toothbrush can reduce the amount of dental plaque.

However, clinical experience seems to indicate that irrespective of daily use of dental floss and/or a toothbrush, dental biofilm remains present on many tooth surfaces.

Moreover, a biofilm matrix such as dental plaque may contribute to the isolation of bacteria from the protective effect of antimicrobial compounds and, thus, may interfere with the function of antimicrobial formulations such as mouthwashes, rinses, and toothpastes.

As a result, alternative methods and compositions to control or prevent the risk of caries are desirable.

In particular, it would be desirable to have a composition which is able to influence metabolic balances residing in oral biofilms which are left in a patient's mouth. Such a composition should be easy in administering and simple in use. Ideally, the composition should be easy to prepare and reasonable in price of manufacturing. Further, the composition should not have undesired side effects like bad taste or being astringent. One or more of the above objects are addressed by the invention described in the present text.

In one embodiment the present invention features a composition, in particular, an oral composition, for use in a method of treating caries by reducing the lactic acid release of lactic acid producing bacteria in an oral biofilm of a human being or animal, the composition comprising
one or more amino acid(s) dissolved or dispersed in water,
the amino acid(s) being selected from glycine, leucine, isoleucine, lysine, methionine, phenylalanine, serine, threonine, valine, tryptophan and mixtures thereof,
   the composition preferably having a pH value in the range of 6-8.

The invention is also related to a kit of parts for use in a method of treating caries by reducing the lactic acid release of lactic acid producing bacteria in an oral biofilm of a human being or animal, the kit of parts containing Part A comprising the amino acid(s) described in the present text Part B comprising water, and optionally Part C comprising an application device.

Described is also a method of using the composition or kit of parts as described in the present text and claims, the method comprises the step of brining the composition in contact with oral biofilm in the mouth of a human being or animal.

The invention is also directed to the use of amino acid(s) selected from glycine, leucine, isoleucine, lysine, methionine, phenylalanine, serine, threonine, valine, tryptophan and mixtures thereof for producing a composition or a kit of parts as described in the present text for use in a method of treating caries by reducing the lactic acid release of lactic acid producing bacteria in an oral biofilm.

Unless defined differently, for this description the following terms shall have the given meaning:

A “composition” is understood to be a mixture of two or more components.

The term “compound” or “component” is a chemical substance which has a particular molecular identity or is made of a mixture of such substances, e.g., polymeric substances.

A “dental or oral care composition” is a composition which is to be used in the dental field (including restorative and prosthodontic work) including the orthodontic area. In this respect the composition should be not detrimental to the patient's health and thus free of hazardous and toxic components being able to migrate out of the composition. Commercially available products have to fulfil certain requirements such as those given in DIN EN ISO 1942:2011-03.

As used herein, a “dental surface” or “dental hard tissue” refers to tooth structures (e. g., enamel, dentin, and cementum) and bone.

A “tooth structure” is any tooth structure, prepared or ready for preparation by the dentist. It can be a single tooth or two or more teeth. A tooth structure is also referred to as hard dental tissue in contrast to soft dental tissue (e.g. gingival).

“Caries” is understood as tooth decay, also known as dental caries which is a demineralization and/or breakdown of teeth due to acids made by bacteria.

“Dental plaque” is understood as is a biofilm or mass of bacteria that grows on surfaces within the mouth.

A “paste” is a substance that behaves as a solid until a sufficiently large load or stress is applied, at which point it flows like a fluid. Pastes typically consist of a suspension of granular material in a background fluid. The individual grains are jammed together like sand on a beach, forming a disordered, glassy or amorphous structure, and giving pastes their solid-like character. Pastes can be classified by their viscosity or their consistency comparable to dental impression material.

A “toothpaste” (dentifrice) is a cleaning agent for the daily individual care. It is typically used as a prophylactic measure against caries, gingivitis or periodontitis. In contrast to this, a “prophylaxis paste” is a product which is used by a profession such as a dentist or a dental hygienist to remove adherent deposits such as stain, plaque or tartar which may stick to the surface of a natural tooth, artificial tooth crown or bridge or filling material. A prophylaxis paste is therefore typically used on slowly rotating paste carrier (sometimes also referred to as prophy cups). Most of the commercially available prophylaxis pastes have a different viscosity compared to tooth pastes.

A “gel” is typically a colloidal system in which a porous matrix of interconnected particles spans the volume of a liquid medium. In general, gels are apparently solid, jelly-like materials. Both by weight and volume, gels are mostly liquid in composition and thus exhibit densities similar to liquids, however, have the structural coherence of a solid. An example of a common gel is edible gelatine. Many gels display thixotropy, that is, they become fluid when agitated, but re-solidify when resting.

A “solvent” means a liquid which is able to at least partially disperse or dissolve a component at ambient conditions (e.g. 23° C.). A solvent typically has a viscosity below 5 or below 1 or below 0.1 Pa*s at 23° C.

A “particle” means a substance being a solid having a shape which can be geometrically determined. Particles can typically be analysed with respect to e.g. grain size.

The mean particle size of a powder can be obtained from the cumulative curve of the grain size distribution and is defined as the arithmetic average of the measured grain sizes of a certain powder mixture. Respective measurements can be done using commercially available granulometers (e.g. CILAS Laser Diffraction Particle Size Analysis Instrument).

“Ambient conditions” mean the conditions which the inventive solution is usually subjected to during storage and handling. Ambient conditions may, for example, be a pressure of 900 to 1100 mbar, a temperature of 10 to 40° C. and a relative humidity of 10 to 100%. In the laboratory ambient conditions are adjusted to 20 to 25° C. and 1,000 to 1,025 mbar.

A composition is “essentially or substantially free of” a certain component, if the composition does not contain said component as an essential feature. Thus, said component is not wilfully added to the composition either as such or in combination with other components or ingredient of other components. A composition being essentially free of a certain component usually does not contain that component at all. However, sometimes the presence of a small amount of the said component is not avoidable e.g. due to impurities contained in the raw materials used.

As used herein, “a”, “an”, “the”, “at least one” and “one or more” are used interchangeably. The terms “comprise” or “contain” and variations thereof do not have a limiting meaning where these terms appear in the description and claims. Also herein, the recitations of numerical ranges by endpoints include all numbers subsumed within that range (e.g., 1 to 5 includes 1, 1.5, 2, 2.75, 3, 3.80, 4, 5, etc.).

The terms “comprise” or “contain” and variations thereof do not have a limiting meaning where these terms appear in the description and claims. The term “comprise” shall include also the terms “consist essentially of” and “consists of”.

“And/or” means one or both. E.g., the expression component A and/or component B refers to a component A alone, component B alone, or to both component A and component B.

Adding an “(s)” to a term means that the term should include the singular and plural form. E.g. the term “additive(s)” means one additive and more additives (e.g. 2, 3, 4, etc.).

Unless otherwise indicated, all numbers expressing quantities of ingredients, measurement of physical properties such as described below and so forth used in the specification and claims are to be understood as being modified in all instances by the term “about”.

### DETAILED DESCRIPTION

It has been found that by application of certain naturally occurring amino acids the lactic-acid release of lactic-acid releasing bacteria in the oral biofilm in the mouth of a living human or animal being can be significantly reduced.

Surprisingly it was found that the amino acids glycine, leucine, isoleucine, lysine, methionine, phenylalanine, serine, threonine, valine, tryptophan are useful for achieving this effect, whereas other amino acids are less effective.

E.g. the amino acid arginine as suggested in WO 2009/100263 A2 (Colgate) and US 2009/0202456 A1 (Prencipe et al.) was found to be not effective.

As the lactic-acid release capacity is correlated with the caries activity of the oral biofilm, the composition described in the present text provides an effective means for reducing the risk of caries.

Unexpectedly, this effect was observed even, if no further active components such as hydroperoxides, hypochlorides, or heavy metal components etc. were present.

Without wishing to be bound to a particular theory, it is assumed that the amino acids described in the present text can influence the metabolic activity of lactic acid releasing bacteria which are present in an oral biofilm of a human or animal being.

It is believed that the amino acids described in the present text are functioning as metabolism modifying agents.

The metabolism modifying agent primarily refers to the metabolism within the cell of the lactic-acid releasing bacteria.

Thus, one aspect of the oral care composition described in the present text is to move from a plaque formation inhibition composition and/or dental plaque removal composition to a composition allowing the control of the metabolic balance on dental plaque.

In particular, it was observed that certain amino acids lower lactic acid release from oral biofilm and lower biofilm mass formation in comparison to control.

It was also observed that certain amino acids lower lactic acid release from oral biofilm without change of biofilm mass formation in comparison to control.

It was also observed that certain amino acids lower lactic acid release from oral biofilm and an increase in biofilm mass formation.

Thus, the release of lactic acid from an oral biofilm caused by the amino acids described in the present text is obviously not correlated with the formation of oral biofilm mass or inhibition of the growing of oral biofilm.

Compared to previously used active components, the amino acids proposed in the present text are naturally occurring substances, easily available at reasonable costs and essentially non-toxic to the patient, even if used at elevated concentrations.

The oral care composition described in the present text for use in a method or therapy of treating caries by reducing the lactic acid release of lactic acid producing bacteria in an oral biofilm of a living human or animal being comprises one or more amino acids. The reduction of the lactic acid release can be determined as described in the example section. However, not all kinds of amino acids were found to be useful, but only a few.

The following amino acids were found to be useful for achieving the desired results: glycine, leucine, isoleucine, lysine, methionine, phenylalanine, serine, threonine, valine, tryptophan and mixtures thereof, with the following amino acids being sometimes preferred: glycine, phenylalanine, serine, isoleucine, leucine, methionine, with glycine and phenylalanine being sometimes being even more preferred. The amino acids may be natural or synthetic. The amino acids might be in D- or L-configuration, wherein the L-configuration is preferred.

In contrast, the following amino acids were found to be not effective for reducing the lactic-acid release of bacteria in a biofilm: proline, arginine, histidine, aspartic acid, glutamine, tyrosine and are therefore not suggested for this particular use.

According to one embodiment the composition described in the present text does typically not contain these amino acids in an effective amount, e.g. more than 0.5 or more than 0.3 or more than 0.1 wt. %.

On the other hand, the amino acids selected from glycine, leucine, isoleucine, lysine, methionine, phenylalanine, serine, threonine, valine, tryptophan and mixtures thereof are present in an amount sufficient for achieving the desired result.

The composition and the amino acids contained therein are used in a therapeutically effective amount being sufficient to influence the lactic acid release metabolism of lactic acid releasing bacteria in an oral biofilm.

The composition and the amino acids contained therein are used for a time period being sufficient to influence the lactic acid release metabolism of lactic acid releasing bacteria in an oral biofilm.

More precisely, the composition and the amino acids contained therein are typically present in an amount and applied for a time period effective to reduce the lactic acid release of lactic acid producing bacteria by more than 40% or more than 45% compared to the situation before the oral care composition described in the present text was used.

A suitable method for determining the effectiveness is described in the example section.

The amino acids are typically present in the following amounts:

Lower limit: at least 0.1 or at least 1 or at least 2 wt. %;

Upper limit: utmost 15 or utmost 12 or utmost 10 wt. %;

Range: 0.1 to 15 or 1 to 12 or 2 to 10 wt. %;

wt. % with respect to the whole amount of the composition.

Depending on the amino acid used certain concentrations might be even more effective than others.

The following concentrations or amounts were found to be particular effective:

glycine in an amount of 0.1 to 10 wt. %; or

leucine in an amount of 0.1 to 5 wt. %; or

isoleucine in an amount of 0.1 to 5 wt. %; or

lysine in an amount of 0.1 to 10 wt. %; or

methionine in an amount of 0.1 to 10 wt. %; or

phenylalanine in an amount of 0.1 to 5 wt. %; or

serine in an amount of 0.1 to 10 wt. %; or

threonine in an amount of 0.1 to 10 wt. %; or

valine in an amount of 0.1 to 8 wt. %; or

tryptophan in an amount of 0.1 to 2 wt. %;

wt. % with respect to the weight of the whole composition.

The following concentrations or amounts were found to be effective, as well:

glycine in an amount of 1 to 10 wt. %; or
leucine in an amount of 1 to 5 wt. %; or
isoleucine in an amount of 2 to 5 wt. %; or
lysine in an amount of 5 to 10 wt. %; or
methionine in an amount of 2 to 10 wt. %; or
phenylalanine in an amount of 1 to 5 wt. %; or
serine in an amount of 1 to 10 wt. %; or
threonine in an amount of 6 to 10 wt. %; or
valine in an amount of 3 to 8 wt. %; or
tryptophan in an amount of 0.5 to 2 wt. %;
wt. % with respect to the weight of the whole composition.

The oral care composition also comprises water.

The water is used for dissolving the amino acids contained in the composition, at least partially.

Water is typically present in an amount sufficient to fully dissolve the amino acids being present in the composition.

It has been found that the desired effect of reducing the lactic-acid release can be achieved more effectively, if the amino acids are present in dissolved form in the composition.

The ratio of amino acid to water is typically at least 0.1/100, or at least 1.5/100, or at least 2/100 with respect to weight.

The ratio of amino acid to water is typically

in a range of 0.1/100 to 15/100,

or in a range of 1.5/100 to 12/100,

or in a range of 2/100 to 10/100 with respect to weight.

The composition may comprise water in the following amounts:

Lower limit: at least 30 or at least 40 or at least 50 wt. %;

Upper limit: utmost 98 or utmost 95 or utmost 90 wt. %;

Range: 30 to 98 or 40 to 95 or 50 to 90 wt. %;

wt. % with respect to the whole amount of the composition.

The composition may also comprise one or more carrier components.

The nature and structure of the carrier component is not particularly limited, unless the desired result cannot be achieved.

Carrier components may help to adjust the rheological properties of the composition.

Different kinds of carrier components can be used, if desired.

Suitable carrier components are gel-forming components or paste-forming components.

Useful examples of gel-forming or paste-forming components include Irish moss, carboxymethyl cellulose, gum tragacanth, gum arabic, gum Karaya, sodium alginate, hydroxyethyl cellulose, methyl and ethyl cellulose, carrageenan, xanthan gum, polyvinyl pyrrolidone, and mixtures thereof.

These gel-forming or paste-forming components do not have abrasive properties with respect to the tooth surface.

If desired, according to a further embodiment, the composition described in the present text may also comprise abrasive particles.

Examples of abrasive particles which may be present include perlite, bentonite, silica, alumina, aluminium hydroxide, ilmenite (FeTiO₃), zircon oxide, zircon silicate, calcium carbonate, sodium bicarbonate, titanium dioxide, precipitated lime, chalk, flour of pumice, zeolites, talcum, kaolin, kieselguhr, aluminium oxide, silicates and mixtures thereof.

If present, the carrier component is typically present in the following amount(s):

Lower limit: at least 1 or at least 2 or at least 5 wt. %;

Upper limit: utmost 50 or utmost 40 or utmost 30 wt. %;

Range: 1 to 50 or 2 to 40 or 5 to 30 wt. %;

wt. % with respect to the whole amount of the composition.

The composition described in the present text may also comprise additive(s). One or more additives can be present, if desired.

If present, additive(s) are typically present in the following amount(s):

Lower limit: at least 0.01 or at least 0.1 or at least 0.5 wt. %;

Upper limit: utmost 10 or utmost 8 or utmost 6 wt. %;

Range: 0.01 to 10 or 0.1 to 8 or 0.5 to 6 wt. %;

wt. % with respect to the whole amount of the composition.

Additive(s) which might be present include stabilizer(s), fluoride releasing agent(s), colourant(s), phosphate releasing agent(s), calcium releasing agent(s), anti-microbial agent(s), buffer(s), surfactant(s), humectant(s) and mixtures thereof.

The composition might contain one or more stabilizer(s) as an additive.

If a stabilizer is present, the storage stability of the dental composition might be improved. That is, the individual components of the composition do not separate over time. A composition is defined as storage-stable, if the components do not separate from each other within about 6 months or about 12 months or about 24 months or about 36 months at ambient conditions.

If a stabilizer is present, it is typically, present in a low amount.

Amounts, found to be useful, include 0.001 to 5 wt. % or 0.01 to 3 wt. % or 0.1 to 1 wt. % with respect to the weight of the whole dental composition. Typical ranges include from 0.01 wt. % to about 5 wt. % or from 0.1 wt. % to 3 wt. % with respect to the amount of the whole composition.

Examples of stabilizer(s) include copolymers of 2,5-furandione with 1,9-decadiene and methoxyethene (e.g. Stabilize™, International Specialty Products (ISP) Comp.) and carboxy vinyl polymers (e.g. Carbopol™, Lubrizol Advanced Materials Comp.).

Stabilizers typically have a mean particle size below 500 μm or below 250 μm or below 100 μm.

In another embodiment, the composition can comprise one or more fluoride releasing agents as an additive.

There is no need for a fluoride releasing agent to be present at all.

However, if it is present, it is typically present in an amount of at most 5 wt. % or of at most 3 wt. % or of at most 2 wt. % with respect to the amount of the whole composition. Typical ranges include from 0.01 wt. % to about 5 wt. % or from 0.1 wt. % to 3 wt. % with respect to the amount of the whole composition.

Examples of fluoride releasing agents include sodium fluoride, potassium fluoride, stannous fluoride, N,N,N′-Tris(2-hydroxyethyl)-N′-octadecyl-1,3-diaminopropan-dihydrofluorid (amine fluoride), sodium monofluorophosphate and mixtures thereof.

In a further embodiment, the composition comprises one or more colourants.

There is no need for a colourant to be present at all.

However, if it is present, it is typically present in an amount of at most 5 wt. % or of at most 3 wt. % or of at most 1 wt. % with respect to the amount of the whole composition. Typical ranges include 0.01 wt. % to 5 wt. % or 0.1 wt. % to 3 wt. % with respect to the amount of the whole composition.

The presence of a colourant may allow an easy detection in a patient's mouth (especially compared to oral tissue and/or tooth substance) and control whether after the treatment all residues of the composition have been removed. E.g., a blue, green or violet colour may be suitable. Colouring of the dental composition can be achieved by incorporating colorants or pigments (organic and inorganic) into the composition.

Examples of colourants include red iron oxide 3395, Bayferrox™ 920 Z Yellow, Neazopon™ Blue 807 (copper phthalocyanine-based dye) or Helio Fast Yellow ER and mixtures thereof.

In a further embodiment, the composition comprises one or more phosphate releasing agent(s) as an additive.

There is no need for a phosphate releasing agent to be present at all.

However, if it is present, it is typically present in an amount of at most 5 wt. % or of at most 3 wt. % or of at most 2 wt. % with respect to the amount of the whole composition. Typical ranges include 0.01 wt. % to 5 wt. % or 0.1 wt. % to 3 wt. % with respect to the amount of the whole composition.

Examples of phosphate and/or calcium releasing agent(s) include calcium pyrophosphate, calcium carbonate, dicalcium phosphate dehydrate, amorphous calcium phosphate, casein phosphopeptide, calcium sodium phosphosilicate, trimetaphosphate, and mixtures thereof.

In another embodiment, the composition comprises anti-microbial agent(s).

There is no need for an anti-microbial agent to be present at all.

However, if it is present, it is typically present in an amount of at most 2 wt. % or of at most 1 wt. % or of at most 0.5 wt. % with respect to the amount of the whole composition. Typical ranges include 0.01 wt. % to 2 wt. % or 0.1 wt. % to 1 wt. % with respect to the amount of the whole composition.

The presence of an anti-microbial agent might help reducing health risks for professionals in the dental offices and laboratories as well as for patients.

Useful anti-microbial agents include chlorhexidine or derivatives thereof and aldehydes (glutaraldeyde, phthalaldehyde) and chlorhexidine or its derivatives and salts of phenolics or acids. It can also be preferred to use acid adducts of chlorhexidine or its derivatives like e.g., acetates, gluconates, chlorides, nitrates, sulphates or carbonates.

Chlorhexidine and its derivatives (hereinafter referred to as CHX) are commercially available in water-based solutions (e.g. a 20% aqueous solution of CHX digluconate, CAS 18472-51-0) or as a pure compound or as a salt. As additive the pure Chlorohexidine compound (CAS 55-56-1) and CHX salts like CHX diacetate monohydrate (CAS 56-95-1) or CHX dihydrochloride (CAS 3697-42-5) are preferred.

CHX also seems to be especially suited as an additive due in part to its well-known and proven anti-microbial action against Gram-positive and Gram-negative microorganisms including the oral Streptococci and Lactobacilli. CHX is bacteriostatic for mycobateria. CHX is also active against yeasts including Candida albicans and viruses including HIV, HBV, HCV, influenza- and herpes virus. A further advantage of CHX is its low toxicity.

Preferred anti-microbial agents include hexitidin, cetypyridiniumcloride (CPC), chlorhexidin (CHX), triclosan, stannous chloride, benzalkonium chloride, non-ionic or ionic surfactants (e.g. quarternary ammonium compounds), alcohols [monomeric, polymeric, mono-alcohols, poly-alcohols (e. g. xylitol, sorbitol), aromatic (e. g. phenol)], antimicrobial peptides (e. g. histatins), bactericins (e. g. nisin), antibiotics (e. g. tetracycline), aldehydes (e. g. glutaraldehyde) inorganic and organic acids (e. g. benzoic acid, salicylic acid, fatty acids) or there salts, derivative of such acids such as esters (e. g. p-hydroxy benzoate or other parabenes, laurizcidin), enzymes (e. g. lysozyme, oxidases), proteins (e. g. enamel matrix protein, proline rich proteins), fluoride, EDTA, essential oils (e. g. thymol).

In another embodiment, the composition can comprise one or more buffer(s) as an additive.

There is no need for a buffer to be present at all.

However, if it is present, it is typically present in an amount of at most 5 wt. % or of at most 3 wt. % or of at most 2 wt. % with respect to the amount of the whole composition. Typical ranges include 0.1 wt. % to 5 wt. % or 1 wt. % to 3 wt. % with respect to the amount of the whole composition.

Examples of buffers, which can be used, include acetic acid/acetate, tris(hydroxymethyl)aminomethane (TRIS), N-(2-acetamido)-2-aminoethane sulfonic acid (ACES), N-(2-acetamido)imminodiacetate (ADA), N,N-bis(2-hydroxyethyl)-2-aminoethane sulfonic acid (BES), 2,2-bis-(hydroxyethyl)-iminotris(hydroxyl-methyl)methane (BIS-TRIS), 2-(cyclohexylamino)ethane sulfonic acid (CHES), 2-[4-(2-hydroxyethyl-1-piperazine)]ethane sulfonic acid (HEPES), 3-[4-(2-hydroxyethyl-1-piperazinyl)]propane sulfonic acid (HEPPS), 2-morpholinoethane sulfonic acid (MES), 3-morpholinopropane sulfonic acid (MOPS), piperazine-1,4-bis(2-ethane sulfonic acid (PIPES), N-[tris(hydroxymethyl)-methyl]-2-aminoethane sulfonic acid (TES), N-[tris(hydroxymethyl)-methyl]-glycine (TRICINE), and phosphate buffers, in particular hydrogen phosphate/dihydrogenphosphate buffers such as Na₂HPO₄, NaH₂PO₄, K₂HPO₄, KH₂PO₄.

In a further embodiment, the dental composition comprises one or more surfactant(s) as an additive.

There is no need for a surfactant to be present at all.

However, if it is present, it is typically present in an amount of at most 5 wt. % or of at most 3 wt. % or of at most 2 wt. % with respect to the amount of the whole composition. Typical ranges include 0.01 wt. % to 5 wt. % or 0.1 wt. % to 3 wt. % with respect to the amount of the whole composition.

Examples of surfactant(s) which can be used include water-soluble salts of alkyl sulphates and alkyl ether sulphates having from 8 to 18 carbon atoms in the alkyl moiety, water-soluble salts of sulfonated monoglycerides of fatty acids having from 8 to 18 carbon atoms in the alkyl radical and mixtures thereof. More specific examples include sodium lauryl sulphate and sodium coconut monoglyceride sulfonates.

The composition might contain one or more humectant(s) as an additive.

There is no need for a humectant to be present at all.

However, if it is present, it is typically present in an amount of at most 5 wt. % or of at most 3 wt. % or of at most 2 wt. % with respect to the amount of the whole composition. Typical ranges include 0.01 wt. % to 5 wt. % or 0.1 wt. % to 3 wt. % with respect to the amount of the whole composition.

Examples of humectant(s) which can be used include glycerine, sorbitol, mannitol, xylitol, propylene glycol, polyethylene glycol and mixtures thereof.

According to one embodiment, the composition comprises:

Component A (amino acid) in an amount of 0.1 to 15 wt. %;

Component B (water) in an amount of 30 to 98 wt. %;

Component C (carrier component) in an amount of 0 to 50 wt. %;

Component D (additive) in an amount of 0 to 10 wt. %;

wt. % with respect to the whole amount of the composition.

According to a further embodiment, the composition comprises:

Component A (amino acid) in an amount of 0.1 to 15 wt. %;

Component B (water) in an amount of 30 to 98 wt. %;

Component C (carrier component) in an amount of 1 to 50 wt. %;

Component D (additive) in an amount of 0 to 10 wt. %;

wt. % with respect to the whole amount of the composition.

According to a further embodiment, the composition comprises:

Component A (amino acid) in an amount of 0.1 to 15 wt. %;

Component B (water) in an amount of 30 to 98 wt. %;

Component C (carrier component) in an amount of 1 to 50 wt. %;

Component D (additive) in an amount of 0.1 to 10 wt. %;

wt. % with respect to the whole amount of the composition.

The composition typically has a pH value in the range of 6 to 8. Thus, the composition is essentially neutral.

The composition can also be characterized by its viscosity. Depending on its chemical formulation, the viscosity may vary over a huge range.

If the composition is provided as liquid, the viscosity is typically in a range of 1 to 10 mPa*s or 1 to 1,000 mPa*s at 23° C.

If the composition is provided as gel or paste, the viscosity is typically in a range of 2,000 to 200,000 mPa*s at 23° C.

If desired, the viscosity of liquids can be measured using a Physica MCR 301 Rheometer (Anton Paar, Graz, Austria) with a cone/plate geometry CP25-1 under controlled shear rate at 23° C. (e.g. 100 s⁻¹). The diameter is 25 mm, the cone angle 1°, and the separation between the cone tip and the plate 49 μm.

If desired, the viscosity of pastes can be determined using a Physica MCR 301 Rheometer (Anton Paar, Graz, Austria) with a plate/plate geometry (PP15) at a constant shear rate of 1 s⁻¹ in rotation at 28° C. The diameter of the plates is 10 mm and the gap between the plates is set to 2.0 mm.

The composition described in the present text can typically be produced as follows:

The amino acid(s) is/are provided and mixed with water, the optional carrier component(s) and the optional additive(s).

Depending on the nature of the optional carrier component(s) and additive(s), the mixing is done by dissolving or dispersing the amino acid(s) in the carrier component(s) and additive(s), if desired, with the aid of a mixing device.

The mixing can be accomplished shortly before the use of the oral care composition or the oral care composition can already be provided in a mixed and storage stable form.

The present invention is also directed to a kit of parts for use in a method or therapy of treating caries by reducing the caries activity or reducing the lactic acid release in an oral biofilm.

The kit of parts typically comprises
Part A comprising the amino acid(s) as described in any the present text (i.e. glycine, leucine, isoleucine, lysine, methionine, phenylalanine, serine, threonine, valine, tryptophan and mixtures thereof),
Part B comprising water for dissolving or dispersing the amino acid(s), optionally in combination with a carrier component, and optionally Part C comprising an application device.

The other components, such as carrier components or additives can be present in Part A or Part B or Part A and Part B, as desired.

Providing the components of the composition in separated parts can be beneficial to improve the storage stability.

Before use, the practitioner will prepare the oral care composition by combining the respective components of the individual parts.

The application of the oral care composition can be done by various means and/or using various devices.

Possible application devices include cups, sponges, brushes, dental trays, syringes, mouth guards, and clear tray aligners.

Clear tray aligners can straighten a dental patient's teeth without the need for using wires and brackets of traditional braces. The aligners typically consist of a sequence of clear, removable trays that fit over the teeth to straighten them.

Using the composition or kit of parts described in the present text in combination with a dental tray, mouth guard or clear tray aligner can be advantageous, as these kinds of devices are typically worn for a longer period of time (e.g. 10 min to 12 hrs) and thus are well suited for applying the oral care composition described in the present text for a longer period of time, if desired.

For use of the composition described in the present text in combination with a dental tray, mouth guard or clear tray aligner, providing the composition in the form of a paste or gel was found to be advantageous.

During storage, the oral care composition described in the present text is typically packaged in a suitable packaging device.

The size and shape of the packaging device typically depends on the form how the composition is provided.

Suitable packaging devices include sealable bottles, tubes, vessels or foil bags (including glass or plastic bottles, e.g. equipped with a screw cap), blisters, syringes, etc.

The packaging device might be designed for single-use or repeated use.

The invention is also related to the use of the amino acid(s) described in the present text for producing an oral care composition or a kit of parts as described in the present text.

The amino acids contained in the oral care composition are used for treating caries by reducing the lactic acid release of lactic acid producing bacteria in an oral biofilm.

The composition or kit described in the present text is designed or intended for use in a method or therapy of reducing the lactic acid release of lactic acid producing bacteria in an oral biofilm of a living human or animal being.

The composition described in the present text may be provided in different forms or shapes.

According to one embodiment, the composition is provided as liquid, e.g. in the form of an oral rinse or a mouth wash.

According to another embodiment, the composition is provided as gel.

According to another embodiment, the composition is provided as paste, e.g. in the form of a tooth paste.

According to another embodiment, the composition is provided as gum, e.g. in the form of a chewing gum.

For reducing the lactic acid release of lactic-acid releasing bacteria in an oral biofilm, the composition has to be brought in contact with the biofilm. The oral biofilm is typically located on tooth surfaces, in particular hard dental tissue.

The bringing into contact can be achieved by different means, including rinsing, spraying, brushing, swabbing, coating or combinations thereof.

The bringing into contact is typically done for a time period being sufficient for causing the desired effect.

The bringing into contact is typically done for a duration of at least 1 min or of at least 2 min or at least 3 min or at least 4 min.

If desired, the step of bringing into contact can be repeated several times.

According to one embodiment, the oral care composition is applied in periodic application scheme.

Possible daily repeating schemes for a periodic application scheme include the following schemes: at least 2 times for at least 1 min within 24 hours; at least 3 times for at least 1 min within 24 hours.

These kinds of repeating schemes are typically applied, if the oral care composition is provided in the form of a mouth wash or tooth paste.

According to one embodiment, the oral care composition is applied in a continuous application scheme.

Possible daily repeating schemes for a continuous application scheme include: at least 1 hour within 24 hours; at least 5 hours within 24 hours.

These kinds of repeating schemes are typically applied, if the oral care composition is provided in the form of a gel or varnish to be applied either directly on the surface of the tooth structure or with the help of an application device.

Both application schemes can be repeated, if desired.

All components used in the dental composition of the invention should be sufficiently biocompatible, that is, the composition should not produce a toxic, injurious, or immunological response in living tissue.

According to certain embodiments, the composition described in the present text does typically not comprise the following components alone or in combination:

oxidizing components in an amount of more than 0.5 wt. %;

heavy metal components comprising Zn or Cu in an amount of more than 0.1 wt. %;

wt. % with respect to the weight of the whole composition.

Certain embodiments of the composition described in the present text are essentially free of abrasive particles, in particular free of the abrasive particles described in the text above. Essentially free means less than 1 or less than 0.5 or less than 0.1 wt. % or do not contain abrasive particles at all. Common to most of these substances is typically a comparable high hardness, e.g. above about Mohs 4 or above about 5.

Certain embodiments of the composition described in the present text are essentially free of oxidizing component(s) (e.g. less than 0.5 or less than 0.3 or less than 0.1 wt. %) or do not contain oxidizing components at all.

Oxidizing component(s) which are typically not present are peroxide, hypochlorite, perborate, persulfate, peroxyphosphate, peroxycarbonate.

Sometimes oxidizing components have a negative effect in that they may react with the amino acids being present in the composition having the result that the reaction products are no longer suitable for acting as a metabolism modifying agent.

Certain embodiments of the composition described in the present text are essentially free of heavy metal component(s), in particular those comprising Zn or Cu (e.g. less than 0.1 or less than 0.05 or less than 0.01 wt. %) or do not contain heavy metal component(s) at all.

Similarly, sometimes heavy metal components may have a negative effect, too, in that they may react with the amino acids being present in the composition (e.g. by forming insoluble complexes) having the result that the reaction products are no longer suitable for acting as a metabolism modifying agent.

However, unavoidable traces of either of these components in the raw materials used for producing the composition may nevertheless be present.

Further embodiments of the invention are given below:

### Embodiment 1

An oral care composition for use in a method of treating caries by reducing the lactic acid release of lactic acid producing bacteria in an oral biofilm of a living human or animal, the composition comprising
amino acids selected from glycine, leucine, isoleucine, lysine, methionine, phenylalanine, serine, threonine, valine, tryptophan and mixtures thereof, water,
   the ratio of amino acid(s) to water being in a range of 0.1 to 100 to 15 to 100 with respect to weight,
   the composition being essentially free of
oxidizing components
heavy metal components comprising Zn or Cu,
   the composition having a pH in the range of 6 to 8 and
   having a viscosity of 1 to 1,000 mPa*s at 23° C.

### Embodiment 2

An oral care composition for use in a method of treating caries by reducing the lactic acid release of lactic acid producing bacteria in an oral biofilm of a living human or animal, the composition comprising
amino acids selected from glycine, leucine, isoleucine, lysine, methionine, phenylalanine, serine, threonine, valine, tryptophan and mixtures thereof, water,
the ratio of amino acid(s) to water being in a range of 1 to 100 to 15 to 100 with respect to weight,
   the composition being essentially free of
oxidizing components
heavy metal components comprising Zn or Cu,
   the composition having a pH in the range of 6 to 8 and
   having a viscosity of 1 to 1,000 mPa*s at 23° C.

### Embodiment 3

An oral care composition for use in a method of treating caries by reducing the lactic acid release of lactic acid producing bacteria in an oral biofilm of a living human or animal being, the composition comprising
amino acids selected from glycine, leucine, isoleucine, lysine, methionine, phenylalanine, serine, threonine, valine, tryptophan and mixtures thereof, water,
a gel-forming or paste-forming component,
   the ratio of amino acid(s) to water being in a range of 0.1 to 100 to 15 to 100 with respect to weight,
   the composition having a pH in the range of 6-8.

### Embodiment 4

An oral care composition for use in a method of treating caries by reducing the lactic acid release of lactic acid producing bacteria in an oral biofilm of a living human or animal being, the composition comprising
amino acids selected from glycine, leucine, isoleucine, lysine, methionine, phenylalanine, serine, threonine, valine, tryptophan and mixtures thereof, water,
a gel-forming or paste-forming component,
   the ratio of amino acid(s) to water being in a range of 0.1 to 100 to 15 to 100 with respect to weight,
   the composition not comprising
oxidizing components in an amount of more than 1 wt. %;
heavy metal components comprising Zn or Cu in an amount of more than 0.1 wt. %;
   the composition having a pH in the range of 6-8 and
   having a viscosity of 2,000 to 20,000 mPa*s at 23° C.

### Embodiment 5

An oral care composition for use in a method of treating caries by reducing the lactic acid release of lactic acid producing bacteria in an oral biofilm of a living human or animal being, the composition comprising
amino acids selected from glycine, leucine, isoleucine, lysine, methionine, phenylalanine, serine, threonine, valine, tryptophan and mixtures thereof, water,
   the ratio of amino acid(s) to water being in a range of 0.1 to 100 to 15 to 100 with respect to weight,
   the composition having a pH in the range of 6-8,
   the method comprising the step of applying the composition to the oral biofilm for at least 1 min.

### Embodiment 6

An oral care composition for use in a method of treating caries by reducing the lactic acid release of lactic acid producing bacteria in an oral biofilm of a living human or animal, the composition comprising

glycine in an amount of 0.1 to 10 wt. %; or

leucine in an amount of 0.1 to 5 wt. %; or

isoleucine in an amount of 0.1 to 5 wt. %; or

lysine in an amount of 0.1 to 10 wt. %; or

methionine in an amount of 0.1 to 10 wt. %; or

phenylalanine in an amount of 0.1 to 5 wt. %; or

serine in an amount of 0.1 to 10 wt. %; or

threonine in an amount of 0.1 to 10 wt. %; or

valine in an amount of 0.1 to 8 wt. %; or

tryptophan in an amount of 0.1 to 2 wt. %; or

mixtures thereof,

water,

wt. % with respect to the weight of the whole composition,
the composition having a pH in the range of 6 to 8,
the method comprising the steps of applying the composition to the oral biofilm according to either of the following application schemes:

at least 2 times for at least 1 min within 24 hours; or

at least 1 hour within 24 hours.

### Embodiment 7

An oral care composition for use in a method of treating caries by reducing the lactic acid release of lactic acid producing bacteria in an oral biofilm of a living human or animal, the composition comprising

glycine in an amount of 0.1 to 10 wt. %; or

leucine in an amount of 0.1 to 5 wt. %; or

isoleucine in an amount of 0.1 to 5 wt. %; or

lysine in an amount of 0.1 to 10 wt. %; or

methionine in an amount of 0.1 to 10 wt. %; or

phenylalanine in an amount of 0.1 to 5 wt. %; or

serine in an amount of 0.1 to 10 wt. %; or

threonine in an amount of 0.1 to 10 wt. %; or

valine in an amount of 0.1 to 8 wt. %; or

tryptophan in an amount of 0.1 to 2 wt. %; or

mixtures thereof,

water,

wt. % with respect to the weight of the whole composition,
the composition having a pH in the range of 6 to 8,
the method comprising the steps of applying the composition to the oral biofilm with the aid of an application device selected from a dental tray, mouth guard or clear tray aligner.

All these compositions can be used in a method or therapy described in the present text.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate, but not limit, the scope of this invention. Unless otherwise indicated, all parts and percentages are by weight.

### EXAMPLES

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Methods

### Amino Acid Containing Compositions

A composition was prepared by dissolving the amino acid to be tested in de-ionized water in the given amount.

### Biofilm Growth and Exposure Procedure

Biofilms were grown in a MCM (mucine containing medium plus 1% sucrose) human derived saliva biofilm system.

A general procedure is summarized below:
1. Four-hour incubation of bovine sample disks with saliva-mcm (mucine containing medium plus 1% sucrose) at 37° C.
2. Fresh medium (MCM with different amino acids) for 2-hour incubation at 37° C.
3. Fresh medium (MCM with different amino acids) for storage over night at 37° C.
4. Second Day: Fresh medium (MCM with different amino acids) after 22 and 24 hours.
5. Samples were taken after 26 hours biofilm growth for measurements of lactic acid release and biomass evaluation.

### Determination of Lactic Acid Release

To determine the activity of the grown biofilm, the production of lactic acid in the supernatant (0.75 ml PBS-solution with 5% sucrose per each sample disc) is measured by using the Lactic acid dehydrogenase (LDH)—Nicotinamid-adenin-dinukleotid (NAD)—Phenazine Methosulphate (PMS)—Thiazolyl blue tetrazolium bromide (MTT)—enzyme assay. To guarantee that only freshly produced lactic acid is determined, the samples are washed (in three steps, each lasting 30 sec) in 1.5 ml PBS-solution. The production of lactic acid is evaluated by measuring the lactic acid amount at given points for 30 min. The determination points are: 0 min 15 min and 30 min. For each reading point a sample of 50 μl is taken out of each well. The lactic acid amount at each reading point is determined by using the enzyme assay described above. Lactic acid amount is calculated according to the lactic acid calibration curve. Instead of lactic acid solution the 504, test aliquots are applied.

### Determination of Mass of Biofilm

The mass of biofilm was determined as follows: Filter papers are put into 1.5 ml-reaction tubes and were labelled and weighted. The biofilm, that was grown on the enamel disks is carefully wiped off with filter paper. The filter paper containing the wet biofilms were placed back into the respective reaction tube and re-weighted. The wet biomass is calculated from the difference in weight.

### Materials

Amino acids and sucrose were obtained from Aldrich.

### Example 1: Influence of Amino Acids on Lactic Acid Release and Biomass Formation

In this experiment, the MCM human derived saliva biofilm system was used. The biofilms were grown for 26 hours in continuous presence of sucrose and different amino acids.

Amino acids have different solubility's in aqueous solution which explains the different amino acid concentrations used.

A reduction of the lactic acid release by more than 40% or more than 45% or more than 50% compared to a control sample was considered to be effective and sufficient.

TABLE 1
influence of amino acid on lactic acid release

| | | Lactic Acid Release | |
|---|---|---|---|
| | Concentration | after 30 min [% | Biomass |
| Amino Acid | [wt. %] | from control] | [% from control] |
| Phenylalanine | 2.7 | 8.6 | 136.4 |
| Proline | 8.0 | 74.7 | 109.9 |
| Serine | 2.7 | 11.8 | 84.6 |
| Threonine | 8.0 | 54.6 | 189.7 |
| Valine | 5.0 | 42.1 | 208.9 |
| Arginine | 8.0 | 99.6 | 247.1 |
| Glycine | 8.0 | 5.7 | 77.2 |
| Histidine | 3.8 | 150.2 | 106.2 |
| Isoleucine | 4.0 | 21.9 | 104.1 |
| Leucine | 2.7 | 36.7 | 85.9 |
| Lysine | 8.0 | 54.7 | 87.8 |
| Methionine | 4.8 | 26.9 | 84.9 |
| Alanine | 4.0 | 99.7 | 140.7 |
| Asparagine | 0.5 | 94.7 | 101.9 |
| Aspartic acid | 0.1125 | 101.0 | 82.1 |
| Cysteine | 0.625 | 85.8 | 56.2 |
| Glutamic acid | 0.215 | 90.4 | 80.2 |
| Tryptophan | 0.25 | 57.7 | 181.0 |
| Tyrosine | 0.0095 | 112.2 | 95.3 |
| Control | — | 100 | 100 |

### Evaluation

After 26-hours growth and continuous presence of glycine (8%) or phenylalanine (2.7%), a reduction of >90% of lactic acid production could be measured.

After 26-hours growth and continuous presence of serine (2.7%), valine (5.0%), isoleucine (4.0%), leucine (2.7%), or methionine (4.8), a reduction of >50% of lactic acid production could be measured.

### Example 2: Glycine Concentration Dependency of Lactic Acid Release Inhibition of Human Saliva Derived Microcosm Biofilm

In this experiment the MCM human derived saliva biofilm system was used.

The biofilms were grown in continuous presence of sucrose and continuous presence of different concentrations of glycine.

TABLE 2
influence of different glycine concentrations on lactic acid release

| | | Lactic Acid Release | |
|---|---|---|---|
| | Concentration | after 30 min [% | Biomass |
| Amino Acid | [wt. %] | from control] | [% from control] |
| Glycine | 8.0 | 5.6 | 42.2 |
| Glycine | 4.0 | 1.6 | 56.2 |
| Glycine | 2.0 | 32.3 | 96.7 |
| Glycine | 1.0 | 51.6 | 145.5 |
| Glycine | 0.5 | 97.5 | 142.7 |
| Control | — | 100.0 | 100.0 |

### Example 3: Phenylalanine Concentration Dependency of Lactic Acid Release Inhibition of Human Saliva Derived Microcosm Biofilm

In this experiment the MCM human derived saliva biofilm system was used. The biofilms were grown in continuous presence of sucrose and different concentrations of phenylalanine.

TABLE 3
influence of different phenylalanine
concentrations on lactic acid release

| | | Lactic Acid Release | |
|---|---|---|---|
| | Concentration | after 30 min [% | Biomass |
| Amino Acid | [wt. %] | from control] | [% from control] |
| Phenylalanine | 2.5 | 3.5 | 66.9 |
| Phenylalanine | 2.0 | 12.1 | 96.5 |
| Phenylalanine | 1.5 | 16.7 | 92.7 |
| Phenylalanine | 1.0 | 32.1 | 124.5 |
| Phenylalanine | 0.5 | 45.9 | 181.4 |
| Control | — | 100.0 | 100.0 |

The changes of lactic acid release from an oral biofilm caused by the amino acids described in the present text is obviously not correlated with the formation of oral biofilm mass or inhibition of the growing of oral biofilm.

1. An oral care composition for use in a method of treating caries by reducing the lactic acid release of lactic acid producing bacteria in an oral biofilm of a human being or animal, the composition comprising:
one or more amino acid(s) dissolved or dispersed in water,
the amino acid(s) being selected from glycine, leucine, isoleucine, lysine, methionine, phenylalanine, serine, threonine, valine, tryptophan and mixtures thereof.

2. The oral care composition for use according to claim 1, the amino acid being present in an amount and applied for a time period effective to reduce the lactic acid release of lactic acid producing bacteria in an oral biofilm by more than 40%.

3. The oral care composition for use according to claim 1 comprising amino acid(s) and water in a ratio of at least 0.1 to 100 with respect to weight.

4. The oral care composition for use according to claim 1 further comprising a carrier component not being water.

5. The oral care composition for use according to claim 4, the carrier component being selected from gel-forming agent(s) and paste-forming agent(s), preferably selected from Irish moss, carboxymethyl cellulose, gum tragacanth, gum arabic, gum Karaya, sodium alginate, hydroxyethyl cellulose, methyl and ethyl cellulose, carrageenan, xanthan gum, polyvinyl pyrrolidone, and mixtures thereof.

6. The oral care composition for use according to claim 1 further comprising additive(s) selected from stabilizer(s), fluoride releasing agent(s), colourant(s), phosphate releasing agent(s), calcium releasing agent(s), anti-microbial agent(s), buffer(s), surfactant(s), humectant(s) and mixtures thereof.

7. The oral care composition for use according to claim 1, the composition being provided in the form of a liquid, gel, paste or gum.

8. The oral care composition for use according to claim 1 not comprising the following components alone or in combination:
oxidizing components in an amount of more than 0.5 wt. %;
heavy metal components comprising Zn or Cu in an amount of more than 0.1 wt. %;
wt. % with respect the weight of the whole composition.

9. The oral care composition for use according claim 1, the reducing of the lactic acid release of lactic-acid releasing bacteria in the oral biofilm being achieved by a process comprising the step of bringing the composition into contact with the oral biofilm for a time sufficient for influencing the metabolic activity of the lactic acid producing bacteria, preferably for a time of at least 1 min.

10. The oral care composition for use according to claim 9, the step of bringing the composition into contact with the oral biofilm being repeated for at least 2 times within 24 hrs.

11. The oral care composition for use according to claim 10, the step of bringing the composition into contact with the oral biofilm being done by rinsing, spraying, brushing, swabbing, coating or combinations thereof.

12. The oral care composition for use in a method of treating caries by reducing the lactic acid release of lactic acid producing bacteria in an oral biofilm of a living human or animal according to any of the preceding claims, the composition comprising
glycine in an amount of 0.1 to 10 wt. %; or
leucine in an amount of 0.1 to 5 wt. %; or
isoleucine in an amount of 0.1 to 5 wt. %; or
lysine in an amount of 0.1 to 10 wt. %; or
methionine in an amount of 0.1 to 10 wt. %; or
phenylalanine in an amount of 0.1 to 5 wt. %; or
serine in an amount of 0.1 to 10 wt. %; or
threonine in an amount of 0.1 to 10 wt. %; or
valine in an amount of 0.1 to 8 wt. %; or
tryptophan in an amount of 0.1 to 2 wt. %; or
mixtures thereof,
water,
the method comprising the steps of applying the composition to the oral biofilm according to either of the following application schemes:
at least 2 times for at least 1 min within 24 hours; or
at least 1 hour within 24 hours.

13. The oral care composition for use according to claim 11, the step of bringing the composition into contact with the oral biofilm being done with the aid of an application device, preferably with the aid of a dental tray, mouth guard or clear tray aligner.

14. (canceled)

15. Use of amino acid(s) selected from glycine, leucine, isoleucine, lysine, methionine, phenylalanine, serine, threonine, valine, tryptophan and mixtures thereof for producing an oral care composition or a kit of parts as described in claim 1 for use in a method of treating caries by reducing the lactic acid release of lactic acid producing bacteria in an oral biofilm of a human being or animal.
